(19) United States
(12) Patent Application Publication
(10) Pub. No.: US 2008/0004281 A1
(43) Pub. Date: Jan. 3, 2008

(54) METHODS FOR THE MODULATION OF CRP BY THE SELECTIVE MODULATION OF PPAR DELTA

(75) Inventors: Tadimeti S. Rao, San Diego, CA (US); John P. McKearn, San Diego, CA (US); Mausumee Guha, San Diego, CA (US)

Correspondence Address: GLOBAL PATENT GROUP - KAL;ATTN: MS LAVERN HALL, 10411 Clayton Road, Suite 304, St. Louis, MO, 63131, US

(73) Assignee: KALYPSYS, INC., San Diego, CA (US)

(21) Appl. No.: 11/768,887

(22) Filed: Jun. 26, 2007

Related U.S. Application Data
(60) Provisional application No. 60/817,571, filed on Jun. 28, 2006.

(51) Int. Cl. A61K 31/495; A61K 31/4985; A61P 19/02

(52) U.S. Cl. 514/249; 514/252.12; 514/255.03

(57) ABSTRACT

The present invention discloses methods to reduce C-reactive protein (CRP) in patients in need thereof comprising the selective modulation of PPARδ.

This application claims the benefit of U.S. Provisional Application No. 60/817,571, filed Jun. 28, 2006, the disclosure of which is hereby incorporated by reference as if written herein in its entirety.

The present invention is directed to methods of reduction of C-reactive protein (CRP) by the selective modulation of the activity of peroxisome proliferator activated receptor-delta (PPARδ), in a subject.

Peroxisome proliferators are a structurally diverse group of compounds which, when administered to mammals, elicit dramatic increases in the size and number of hepatic and renal peroxisomes, as well as concomitant increases in the capacity of peroxisomes to metabolize fatty acids via increased expression of the enzymes required for the β-oxidation cycle (Lazarow and Fujiki, Ann. Rev. Cell Biol. 1:489-530 (1985); Vamecq and Draye, Essays Biochem. 24:1115-225 (1989); and Nelali et al., Cancer Res. 48:5316-5324 (1988)). Compounds that activate or otherwise interact with one or more of the PPARs have been implicated in the regulation of triglyceride and cholesterol levels in animal models. Compounds included in this group are the fibrate class of hypolipidemic drugs, herbicides, and phthalate plasticizers (Reddy and Lalwani, Crit. Rev. Toxicol. 12:1-58 (1983)). Peroxisome proliferation can also be elicited by dietary or physiological factors such as a high-fat diet and cold acclimatization.

Biological processes modulated by PPAR are those modulated by receptors, or receptor combinations, which are responsive to the PPAR receptor ligands. These processes include, for example, plasma lipid transport and fatty acid catabolism, regulation of insulin sensitivity and blood glucose levels, which are involved in hypoglycemia/hyperinsulinemia (resulting from, for example, abnormal pancreatic beta cell function, insulin secreting tumors and/or autoimmune hypoglycemia due to autoantibodies to insulin, the insulin receptor, or autoantibodies that are stimulatory to pancreatic beta cells), macrophage differentiation which lead to the formation of atherosclerotic plaques, carcinogenesis, hyperplasia, and adipocyte differentiation. Additionally, recent evidence points to a role for PPARδ in the development of cancers, including colon, skin, and lung cancers. Modulators of PPAR could therefore find use in the treatment of cancers of various types.

Subtypes of PPAR include PPARα, PPARδ (also known as NUC1, PPARβ and FAAR) and two isoforms of PPARγ. These PPARs can regulate expression of target genes by binding to DNA sequence elements, termed PPAR response elements (PPRE). To date, PPRE's have been identified in the enhancers of a number of genes encoding proteins that regulate lipid metabolism suggesting that PPARs play a pivotal role in the adipogenic signaling cascade and lipid homeostasis (H. Keller and W. Wahli, Trends Endoodn. Met. 291-296, 4 (1993)).

Insight into the mechanism whereby peroxisome proliferators exert their pleiotropic effects was provided by the identification of a member of the nuclear hormone receptor superfamily activated by these chemicals (Isseman and Green, Nature 347-645-650 (1990)). The receptor, termed PPARα, was subsequently shown to be activated by a variety of medium and long-chain fatty acids and to stimulate expression of the genes encoding rat acyl-CoA oxidase and hydratase-dehydrogenase (enzymes required for peroxisomal β-oxidation), as well as rabbit cytochrome P450 4A6, a fatty acid ω-hydroxylase (Gottlicher et al., Proc. Natl. Acad. Sci. USA 89:4653-4657 (1992); Tugwood et al., EMBO J. 11:433-439 (1992); Bardot et al., Biochem. Biophys. Res. Comm. 192:37-45 (1993); Muerhoff et al., J. Biol. Chem. 267:19051-19053 (1992); and Marcus et al., Proc. Natl. Acad. Sci. USA 90(12):5723-5727 (1993)).

Activators of the nuclear receptor PPARγ, for example troglitazone, have been clinically shown to enhance insulin-action, to reduce serum glucose and to have small but significant effects on reducing serum triglyceride levels in patients with Type 2 diabetes. See, for example, D. E. Kelly et al., Curr. Opin. Endocrinol. Diabetes, 90-96, 5 (2), (1998); M. D. Johnson et al., Ann. Pharmacother., 337-348, 32 (3), (1997); and M. Leutenegger et al., Curr. Ther. Res., 403-416, 58 (7), (1997).

The third subtype of PPAR, PPARδ (or alternatively, FAAR, PPARβ or NUC1) initially received much less attention than the other PPARs because of its ubiquitous expression and the unavailability of selective ligands. However, genetic studies and recently developed synthetic PPARδ agonists have helped reveal its role as a powerful regulator of fatty acid catabolism and energy homeostasis. Studies in adipose tissue and muscle have begun to uncover the metabolic functions of PPARδ. Transgenic expression of an activated form of PPARδ in adipose tissue produces lean mice that show enhanced fatty acid oxidation and are resistant to obesity, hyperlipidemia and tissue steatosis induced genetically or by a high-fat diet (Wang Y X et al. Cell 2003:113:159-70). The activated receptor induces genes required for fatty acid catabolism and adaptive thermogenesis. Interestingly, the transcription of PPARγ target genes for lipid storage and lipogenesis remain unchanged. In parallel, PPARδ-deficient mice challenged with a high-fat diet show reduced energy uncoupling and are prone to obesity. Together, these data identify PPARδ as a key regulator of fat-burning, a role that opposes the fat-storing function of PPARγ. In skeletal muscle, PPARδ likewise upregulates fatty acid oxidation and energy expenditure, to a far greater extent than does the lesser-expressed PPARα (Evans R M et al 2004 Nature Med 1-7, 10 (4), 2004). Thus, despite their close evolutionary and structural kinship, PPARδ and the other isoforms, PPARγ and PPARα, regulate distinct genetic networks.

PPARδ is broadly expressed in the body and has been shown to be a valuable molecular target for treatment of dyslipidemia and other diseases. For example, in a recent study in insulin-resistant obese rhesus monkeys, a potent and selective PPARδ compound was shown to decrease VLDL and increase HDL in a dose response manner (Oliver et al., Proc. Natl. Acad. Sci. U.S.A. 98: 5305, 2001). Also, in a recent study in wild-type and HDL-lacking, ABCA1^{-/-} mice, a different potent and selective PPARδ compound was shown to reduce fractional cholesterol absorption in the intestine, and coincidentally reduce expression of the cholesterol-absorption protein NPC1L1 (van der Veen et al., J. Lipid Res. 2005 46: 526-534).

Epidemiological studies suggest that C-reactive protein (CRP) is a strong predictor of cardiovascular risk (Ridker P. Circulation. 2003; 107:363-369). CRP may not only act as a predictor or cardiovasuclar risk but it may also cause diseases such as cardiovascular disease. Even slight increases in CRP without clinical indications of disease correlate with considerably increased cardio- and cerebrovascular morbidity and mortality (Ridker et al, N Engl J Med 1997; 336: 973-9; Haverkate et al, for the European Concerted Action on Thrombosis and Disabilities Angina Pectoris Study Group, The Lancet 1997; 349: 462-6; Harris et al, Am J Med 1999; 106: 506-12; Ridker et al, N Engl J Med 2002; 347 (20): 1557-65). CRP is an acute phase protein produced by the hepatocyte upon stimulation by proinflammatory cytokines, particularly interleukin 6. CRP serum levels increase rapidly in response to infections, tissue damage, and several other inflammatory stimuli. High levels of CRP have been reported in patients with sepsis, rheumatoid arthritis, and conditions associated with tissue damage. CRP levels return to normal gradually upon resolution of the inflammatory cascade. Within individuals, CRP levels remain relatively constant over time, which makes it possible to monitor inflammatory responses based on serial CRP measurements. In the presence of sepsis and active inflammatory conditions such as Crohn's disease and rheumatoid arthritis, serum CRP can reach very high levels, eg, 300 mg/L. In contrast, levels associated with an increased risk of developing coronary artery disease or cardiovascular events are very modest in comparison, eg, 3-10 mg/L (Ridker, 2003). Therefore, high sensitivity CRP (Hs-CRP) assays have been developed to examine moderate yet clinically relevant increases in CRP.

The role of CRP as a component of the innate immune system has led researchers to suggest a pathogenic role for this acute phase protein in atherosclerosis (Li J J, Fang C H. Med Hypotheses. 2004; 62:499-506. Pasceri V et al. Circulation. 2000; 102:2165-2168). Strong evidence exists that CRP levels are associated with different forms of cardiovascular disease, including chronic stable angina, acute coronary syndrome, peripheral vascular disease, and stroke (Ridker, 2003). CRP has been found in active atherosclerotic plaques, where its accumulation antedates that of activated macrophages. CRP activates different complement fractions and colocalizes with the membrane attack complex in early atheromatous lesions (Torzewski M et al. Arterioscler Thromb Vasc Biol. 2000; 20:2094-2099); (ii) CRP stimulates tissue factor production; (iii) CRP stimulates the production and release of proinflammatory cytokines, and increases expression of adhesion molecules; (iv) CRP mediates the uptake of low-density lipoprotein (LDL) cholesterol by macrophages. In addition, the infusion of recombinant CRP activates inflammation and coagulation in humans (Bisoendial R J et al. Circ Res. 2005; 96:714-716), thus endorsing the hypothesis that CRP may have a pathogenic role in cardiovascular disease.

A novel method for the reduction of CRP comprising the selective modulation of PPARδ activity has been discovered and is herein disclosed. In certain embodiments, said modulation is at least 100-fold selective for PPARδ over PPARγ and PPARα.

In further embodiments, the selective modulation of PPARδ over PPARγ and PPAR is over 500-fold selective. In yet further embodiments, said modulation is over 1000-fold selective.

The attenuation of CRP levels through modulation of PPARδ represents a heretofore unknown pharmacologic intervention strategy for the treatment of inflammatory diseases. Inflammatory diseases which may be ameliorated by the reduction of CRP via selective modulation of PPARδ include, but are not limited to, diabetes, heart disease, atherosclerosis, atherothrombosis, Alzheimer's Disease (AD), and diabetic neuropathy.

In one embodiment, the present invention uses a certain compounds from a class of sulfonyl-substituted bicyclic compounds, which are defined by structural Formula I, to modulate CRP levels:

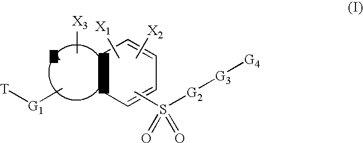

or a salt, ester, or prodrug thereof, wherein;

A is a saturated or unsaturated hydrocarbon chain or a heteroatom-comprising hydrocarbon chain having from 3 to 5 atoms, forming a five- to seven-membered ring;

T is selected from the group consisting of —C(O)OH, —C(O)NH₂, and tetrazole;

G₁ is selected from the group consisting of —(CR¹R²)ₙ—, -Z(CR¹R²)ₙ—, —(CR¹R²)ₙZ-, —(CR¹R²)ᵣZ(CR¹R²)ₛ—;

Z is O, S or NR;

n is 0, 1, or 2;

r and s are independently 0 or 1;

R¹ and R² are independently selected from the group consisting of hydrogen, halo, optionally substituted lower alkyl, optionally substituted lower heteroalkyl, optionally substituted lower alkoxy, and lower perhaloalkyl or together may form an optionally substituted cycloalkyl;

X₁, X₂, and X₃ are independently selected from the group consisting of hydrogen, optionally substituted lower alkyl, optionally substituted cycloalkyl, halogen, perhaloalkyl, hydroxy, optionally substituted lower alkoxy, nitro, cyano, and NH₂;

G₂ is selected from the group consisting of a saturated or unsaturated cycloalkyl or heterocycloalkyl linker, optionally substituted with X₄ and X₅;

X₄ and X₅ are independently selected from the group consisting of hydrogen, optionally substituted lower alkyl, halogen, lower perhaloalkyl, hydroxy, optionally substituted lower alkoxy, nitro, cyano, NH₂, and CO₂R;

R is selected from the group consisting of optionally substituted lower alkyl and hydrogen;

G₃ is selected from the group consisting of a bond, a double bond, —(CR³R⁴)ₘ—, carbonyl, and —(CR³R⁴)ₘCR³═CR⁴—;

m is 0, 1, or 2;

R³ and R⁴ are independently selected from the group consisting of hydrogen, optionally substituted lower alkyl, optionally substituted lower alkoxy, optionally substituted aryl, lower perhaloalkyl, cyano, and nitro;

G₄ is selected from the group consisting of hydrogen, optionally substituted aryl, optionally substituted heteroaryl, optionally substituted cycloalkyl, optionally substituted cycloheteroalkyl, optionally substituted cycloheteroaryl, optionally substituted cycloalkenyl, and —N═(CR⁵R⁶); and

R⁵ and R⁶ are independently selected from the group consisting of hydrogen, optionally substituted aryl, optionally substituted heteroaryl, optionally substituted cycloalkyl, optionally substituted cycloalkenyl, and optionally substituted cycloheteroalkyl.

In further embodiments, the compound of Formula I has the structural formula

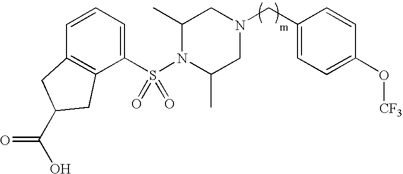

and m is 0 or 1.

In yet further embodiments, the compound is selected from the group consisting of Compound 1, Compound 1A, Compound 3, and Compound 3A, described below.

In yet further embodiments, the compound is Compound 1A.

In a related embodiment, also contemplated herein is a method of reducing CRP comprising the administration of a therapeutically effective amount of (S)-4-[cis-2,6-dimethyl-4-(4-trifluoromethoxy-phenyl)-piperazine-1-sulfonyl]-indan-2-carboxylic acid tosylate, described below.

In further embodiments, said effective amount is greater than 20 mg per day when dosed orally. In yet further embodiments, said amount is between about 20 and about 140 mg per day when dosed orally. In yet further embodiments, said amount is between about 20 and about 80 mg per day when dosed orally. In yet further embodiments, said amount is between about 40 and about 80 mg per day when dosed orally. In certain embodiments, said effective amount, when dosed orally, is selected from the group consisting of: about 20 mg per day, about 40 mg per day, about 60 mg per day, about 80 mg per day, and about 100 mg per day.

In other embodiments, the present invention provides pharmaceutical composition comprising:

one or more selective PPARδ modulators;

an amount of a folate effective to reduce CRP; and

together with one or more pharmaceutically acceptable active ingredients or adjuvants.

In further embodiments, said folate is folic acid.

In another embodiment, also contemplated herein is a method of treating a patient, comprising:

determining CRP levels of said patient;

determining if said CRP levels are too high for said patient; and

if said CRP levels are too high, having said patient take a therapeutically effective amount of a selective PPARδ modulator.

As used herein, the terms below have the meanings indicated.

When ranges of values are disclosed, and the notation “from n₁ . . . to n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—), (—C::C—)]. Examples of suitable alkenyl radicals include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to 20 carbon atoms. In certain embodiments, said alkyl will comprise from 1 to 10 carbon atoms. In further embodiments, said alkyl will comprise from 1 to 6 carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. The term “alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(═O)—NR₂ group with R as defined herein. The term “N-amido” as used herein, alone or in combination, refers to a RC (═O)NH— group, with R as defined herein. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′, wherein R and R′ are independently selected from the group consisting of hydrogen, alkyl, acyl, heteroalkyl, aryl, cycloalkyl, heteroaryl, and heterocycloalkyl, any of which may themselves be optionally substituted.

The term “aryl,” as used herein, alone or in combination, means a carbocyclic aromatic system containing one, two or three rings wherein such rings may be attached together in a pendent manner or may be fused. The term “aryl” embraces aromatic radicals such as benzyl, phenyl, naphthyl, anthracenyl, phenanthryl, indanyl, indenyl, annulenyl, azulenyl, tetrahydronaphthyl, and biphenyl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, napthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄=derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group-with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl radical wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. Examples of such cycloalkyl radicals include cyclopropyl, cyclobutyl, cyclopentyl, cyclohexyl, cycloheptyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃—.

The term “heteroaryl,” as used herein, alone or in combination, refers to 3 to 7 membered unsaturated heteromonocyclic rings, or fused polycyclic rings in which at least one of the fused rings is unsaturated, wherein at least one atom is selected from the group consisting of O, S, and N. In certain embodiments, said heteroaryl will comprise from 5 to 7 carbon atoms. The term also embraces fused polycyclic groups wherein heterocyclic radicals are fused with aryl radicals, wherein heteroaryl radicals are fused with other heteroaryl radicals, or wherein heteroaryl radicals are fused with cycloalkyl radicals. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic radical containing at least one heteroatom as ring members, wherein each said heteroatom may be independently selected from the group consisting of nitrogen, oxygen, and sulfur In certain embodiments, said heterocycloalkyl will comprise from 1 to 4 heteroatoms as ring members. In further embodiments, said heterocycloalkyl will comprise from 1 to 2 heteroatoms ring members. In certain embodiments, said heterocycloalkyl will comprise from 3 to 8 ring members in each ring. In further embodiments, said heterocycloalkyl will comprise from 3 to 7 ring members in each ring. In yet further embodiments, said heterocycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Heterocycle groups of the invention are exemplified by aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of this invention.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in combination, means containing from 1 to and including 6 carbon atoms.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds of the present invention. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and l-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds of the present invention may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

In the event that any element is designated to be “a bond,” what is meant is that said element collapses to a bond linking the elements on both its sides. For example, in Formula I above, when G₃ is designated to be “a bond”, the structure shown below (right side) is intended: the entity designated G₃ collapses to a bond connecting G₂ and G₄:

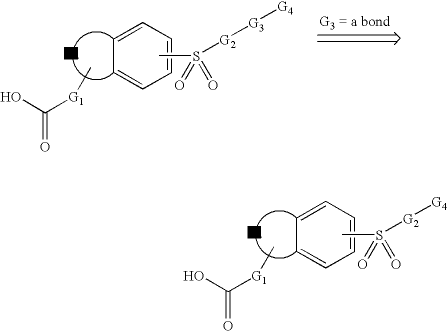

### Similarly, when, within G₁, n is 0 or both r and s are 0, G₁ collapses to a bond connecting A and T.

The term “disease” as used herein is intended to be generally synonymous, and is used interchangeably with, the terms “disorder” and “condition” (as in medical condition), in that all reflect an abnormal condition of the body or of one of its parts that impairs normal functioning and is typically manifested by distinguishing signs and symptoms.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The term “activate” refers to increasing the cellular function of a target enzyme or protein.

The term “inhibit” refers to decreasing the cellular function of a target enzyme or protein. The target enzyme or protein function may be the interaction with a natural binding partner or catalytic activity.

The term “modulate” refers to the ability of a compound of the invention to alter the function of a target enzyme or protein. A modulator may activate the activity of a target enzyme or protein. The term “modulate” also refers to altering the function of a target enzyme or protein by increasing or decreasing the probability that a complex forms between a target enzyme or protein and a natural binding partner. A modulator may increase the probability that such a complex forms between the target enzyme or protein and the natural binding partner, may increase or decrease the probability that a complex forms between the target enzyme or protein and the natural binding partner depending on the concentration of the compound exposed to the target enzyme or protein, and or may decrease the probability that a complex forms between the target enzyme or protein and the natural binding partner.

The term “selective” as used herein means having the characteristic or property of being highly specific in binding, activity, or effect. Compounds described herein as selective for PPARδ over other isoforms, for example, preferentially bind and/or modulate PPARδ in favor of PPARγ and PPARα. The degree of selectivity may vary, but in many embodiments, a selective compound would be at least tenfold selective for the desired target (e.g., PPARδ). In certain embodiments, the compound would be 100- to 1000-fold selective. Alternatively, a compound may be selective the sense of producing a differential effect. For example, such a compound may bind both PPARδ and PPARγ with equal or similar affinity, but activate one while inhibiting the other. Such a mechanism has not been described at this time, but, not wishing to be bound by theory, it is postulated here.

“PPARδ modulator” is used herein to refer to a compound that exhibits an EC₅₀ with respect to PPARδ activity of no more than about 100 μM and more typically not more than about 50 μM, as measured in the PPARδ assay described generally hereinbelow. “EC₅₀” is that concentration of modulator which either activates or reduces the activity of an enzyme (e.g., PPARδ) to half-maximal level. Certain representative compounds of the present invention have been discovered to exhibit modulatory activity against PPARδ. In certain embodiments, compounds will exhibit an EC₅₀ with respect to PPARδ of no more than about 10 μM; in further embodiments, compounds will exhibit an EC₅₀ with respect to PPARδ of no more than about 5 μM; in yet further embodiments, compounds will exhibit an EC₅₀ with respect to PPARδ of not more than about 1 μM; in yet further embodiments, compounds will exhibit an EC₅₀ with respect to PPARδ of not more than about 200 nM, as measured in the PPARδ assay described herein.

The term “therapeutically effective amount” as used herein refers to that amount of the compound being administered which will relieve to some extent one or more of the symptoms of the disease, condition or disorder being treated.

The term “prodrug” refers to a compound that is made more active in vivo. Certain compounds of the present invention may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds of the present invention can exist as therapeutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds of the present invention which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

In certain embodiments, the salt may be selected from the group consisting of the hydrochloride, hydrobromide, sulfonate, citrate, tartrate, phosphonate, lactate, pyruvate, acetate, succinate, oxalate, fumarate, maleate, oxaloacetate, methanesulfonate, ethanesulfonate, p-toluenesulfonate (tosylate), benzenesulfonate (besylate) and isethionate salts of compounds, including (S)-4-[cis-2,6-dimethyl-4-(4-trifluoromethoxy-phenyl)-piperazine-1-sulfonyl]-indan-2-carboxylic acid and (S)-4-[cis-2,6-dimethyl-4-(4-trifluoromethoxy-benzyl)-piperazine-1-sulfonyl]-indan-2-carboxylic acid. In further embodiments, the salt is the tosylate salt.

Salts of compounds of the present invention may be formed by contacting said compounds with an appropriate acid or counterion, either neat or in a suitable solvent. For example, compounds of Formula I can be contacted with p-toluenesulfonic acid to yield the p-toluenesulfonate (tosylate) salt form of the invention. Compounds of Formula I, including Compounds 1, 1A, 3, and 3A below, their racemates, and racemic mixtures thereof, prepared by any method can be contacted with a reagent selected from the group consisting of calcium acetate, hydrochloric acid, phosphoric acid, sulfuric acid, sodium hydroxide, potassium hydroxide, magnesium acetate, and p-toluenesulfonic acid, for example in a 1:1 ratio, in a suitable solvent. Such solvents include but are not limited to diisopropyl ether, toluene, dichloromethane, and acetonitrile. Any technique known in the art can be used to vary conditions to induce precipitation or crystallization, including, without limitation: stirring for varying lengths of time at varying ambient conditions, the addition of hexanes or diethyl ether, evaporation, and reduction of temperature. In particular, 4-[cis-2,6-dimethyl-4-(4-trifluoromethoxy-phenyl)-piperazine-1-sulfonyl]-indan-2-carboxylic acid can be contacted with p-toluenesulfonic acid to yield 4-[cis-2,6-dimethyl-4-(4-trifluoromethoxy-phenyl)-piperazine-1-sulfonyl]-indan-2-carboxylic acid tosylate. The present invention provides for salts of each racemate of compounds of Formula I, including (S)-4-[cis-2,6-dimethyl-4-(4-trifluoromethoxy-phenyl)-piperazine-1-sulfonyl]-indan-2-carboxylic acid tosylate. Additional methods of forming salts of compounds of Formula I are described below, and may also be found in U.S. patent application Ser. No. 11/552,134, filed Oct. 23, 2006, the disclosure of which is hereby incorporated by reference as if written herein in its entirety.

While it may be possible for the compounds, or their salts, esters, prodrugs, amides, or solvates, to be administered as raw chemical, it is also possible to present them as a pharmaceutical formulation. Accordingly, provided herein are pharmaceutical formulations which comprise one or more of certain compounds of the present invention, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing into association a compound of the subject invention or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds of the present invention may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

In certain embodiments, the compound may be formulated as a tablet or capsule.

In further embodiments, the compound will be a compound of Formula I.

In yet further embodiments, the compound will be Compound 1A.

In certain embodiments, the formulation will also comprise one or more fillers, such as lactose monohydrate, microcrystalline cellulose or Prosolv; one or more disintegrating agents such as povidone or crospovidone; and one or more lubricants such as magnesium stearate. In yet further embodiments, the formulation will also comprise one or more glidants, such as silicon dioxide or Lutrol.

In certain embodiments, formulations comprising compounds as described herein will be capsules which may comprise:
a. 0.2-20% (S)-4-[cis-2,6-dimethyl-4-(4-trifluoromethoxy-phenyl)-piperazine-1-sulfonyl]-indan-2-carboxylic acid tosylate, the particle size thereof ranging from about 50 to about 250 microns;
b. 0-99.99% lactose monohydrate;
c. 0-10% crospovidone;
d. 0-5% magnesium stearate; and
e. 0-5% silicon dioxide.

Yet further embodiments of formulations of compounds of the present invention presented as formulations may be found below.

Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of the compounds of the invention with: (a) statin and/or other lipid lowering drugs for example MTP inhibitors and LDLR upregulators; (b) antidiabetic agents, e.g. metformin, sulfonylureas, or PPAR-gamma, PPAR-alpha and PPAR-alpha/gamma modulators (for example thiazolidinediones such as e.g. Pioglitazone and Rosiglitazone, fibrates, and niacin); and (c) antihypertensive agents such as angiotensin antagonists, e.g., telmisartan, calcium channel antagonists, e.g. lacidipine and ACE inhibitors, e.g., enalapril.

In another embodiment, the present invention comprises administering PPARδ compounds together with folates. The folates which can be used include both pteroic acid monoglutamate (folic acid) and reduced forms such as dihydrofolates and tetrahydrofolates, polyglutamates thereof, optical isomers thereof and pharmaceutically acceptable salts thereof. In certain embodiments, the folates which are used are tetrahydrofolates; in further embodiments, the folates are natural stereoisomeric forms of tetrahydrofolates such as 5-formyl-(6S)-tetrahydrofolic acid, 5-methyl-(6S)-tetrahydrofolic acid, 5,10-methylene-(6R)-tetrahydrofolic acid, 5,10-methenyl-(6R)-tetrahydrofolic acid, 10-formyl-(6R)-tetrahydrofolic acid, 5-formimino-(6S)-tetrahydrofolic acid or (6S)-tetrahydrofolic acid or pharmaceutically acceptable salts thereof. The folates which are used can generally be converted into one another by folate metabolism. In certain embodiments, 5-methyl-(6S)-tetrahydrofolic acid, 5-formyl-(6S)-tetrahydrofolic acid and pharmaceutically acceptable salts thereof are used (see US2006/0063768A1).

In any case, the multiple therapeutic agents (at least one of which is a PPARδ modulator) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, the present invention provides methods for treating PPAR-mediated disorders in a human or animal subject in need of such treatment comprising administering to said subject an amount of a compound of the present invention effective to reduce or prevent said disorder in the subject in combination with at least one additional agent for the treatment of said disorder that is known in the art. In a related aspect, the present invention provides therapeutic compositions comprising at least one compound of the present invention in combination with one or more additional agents for the treatment of PPAR-mediated disorders.

PPAR delta modulators, by virtue of lowering CRP levels, have the potential to treat a number of clinical conditions where vascular inflammation is a key contributor. Diseases, disorders, and conditions which may be ameliorated by methods and compositions disclosed herein include, without limitation: inflammatory disorders of the cardiovascular system, such as atherosclerosis, vascular inflammation, coronary artery disease, peripheral arterial and/or vascular disease, chronic intermittent claudication and thrombosis, any of which may or may not be associated with or complications arising from diabetes; metabolic inflammatory disorders such as diabetes and co-morbid inflammation associated with obesity; ischemic conditions such as stroke, aortic aneurysm, and transient ischemic attacks; complement factor diseases including those associated with complement factor H, for example, thrombotic microangiopathies, glomerulonephritides, hemolytic uremic syndrome (HUS), membranoproliferative glomerulonephritis (dense deposit disease), age-related macular degeneration and chronic conditions featuring debris deposition; symptoms associated with menopause, particularly in women receiving hormone therapy; and renal and nephritic disorders such as chronic kidney failure. Methods and compositions disclosed herein may also be employed in conjunction with therapies to treat the aforementioned disorders. For example, methods and compositions disclosed herein may be ased as adjuvant therapy to hemodialysis or peritoneal dialysis for the treatment of renal diseases, or as adjuvant therapy in patients undergoing stent implantation surgery for stenosis or percutaneous transluminal angioplasty.

In yet another embodiment the present invention provides a method of treating a patient by first testing for CRP levels using methods well known in the art (such as the method disclosed in US20060029976A1), second determining if the patient CRP levels are too high and then third if the levels are too high then having the patient take PPARδ compounds to lower the CRP levels.

Besides being useful for human treatment, the compounds and formulations of the present invention are also useful for veterinary treatment of companion animals, exotic animals and farm animals, including mammals, rodents, and the like. More preferred animals include horses, dogs, and cats.

Other compounds which may be used according to the methods disclosed herein include certain of those described in: PCT/US2004/010737, filed on Apr. 7, 2004; PCT/US2004/010889, filed on Apr. 7, 2004; PCT/US2004/010970, filed on Apr. 7, 2004; U.S. application Ser. No. 10/820,647, filed Apr. 7, 2004; PCT/US2004/043031, filed Dec. 20, 2004; PCT/US2005/011751 filed Apr. 7, 2005; U.S. application Ser. No. 11/102,356 filed Apr. 7, 2005; U.S. application Ser. No. 11/258,463, filed Oct. 25, 2005; and U.S. Pat. No. 6,710,063, issued Mar. 23, 2004; the contents of all of which are hereby incorporated by reference in their entirety.

The invention is further illustrated by the following examples.

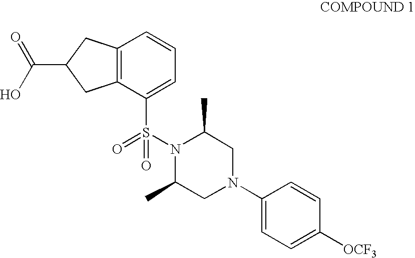

### 4-[cis-2,6-Dimethyl-4-(4-trifluoromethoxy-phenyl)-piperazine-1-sulfonyl]-indan-2-carboxylic acid

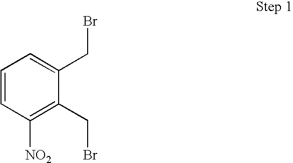

1,2-Bis(bromomethyl)-3-nitrobenzene: A 1 liter flask was charged with 1,2-dimethyl-3-nitrobenzene (20 g, 0.13 mol), N-bromosuccinimide (50 g, 0.28 mol), azobis(isobutyronitrile) (5 g, 3.0 mmol), and 200 mL of dichloromethane. This was irradiated with a 120 watt floodlamp to affect gentle reflux under nitrogen for 18 hours. The mixture was then cooled and precipitated succinimide was removed by filtration. The filtrate was concentrated and the residue was purified by chromatography on silica (5%-50% CH₂Cl₂ in hexanes) to give 2.6 g white solid (64%).

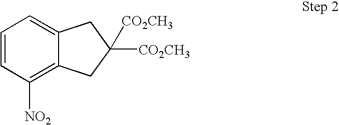

Dimethyl-4-nitroindane-2,2-dicarboxylate: To a solution stirred under nitrogen at room temperature, to 5.0 mL methanol in 15.0 mL ether was added 60% sodium hydride (0.84 g, 0.021 mol) in small portions. After the addition was complete, the nearly clear and colorless solution was stirred for 5 minutes. To it was then added 1.3 g dimethyl malonate, giving a slightly cloudy colorless solution. To this was rapidly added a suspension of 3.1 g 1,2-bis(bromomethyl)3-nitrobenzene, which immediate gave a precipitate suspended in a dark green solution. This was removed by filtration and the filtrate was concentrated. The residue was purified on silica (20%-100% CH₂Cl₂ in hexanes) to give 1.93 g off-white solid (67%).

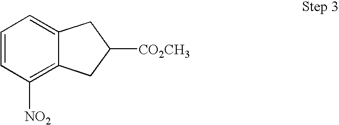

Methyl-4-nitroindane-2-carboxylate: A mixture of dimethyl-4-nitroindane-2,2-dicarboxylate (4.84 g, 0.0167 mol), lithium chloride (0.84 g, 0.0198 mol), 1.11 mL water, and 18 mL dimethylsulfoxide was heated to 160° C. under nitrogen for two hours. It was then allowed to cool and the dimethylsulfoxide was removed under high vacuum. The residue was purified on silica (10%-100% CH₂Cl₂ in hexanes) to give 2.5 g white solid (65%).

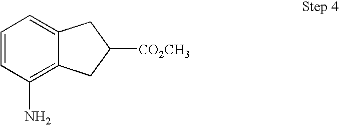

Methyl-4-aminoindane-2-carboxylate: A mixture of methyl-4-nitroindane-2-carboxylate (2.4 g, 0.11 mol) and 10% palladium on carbon (1.1 g, 0.01 mol) in ethyl acetate (15 mL) was shaken under 55 PSI hydrogen for 1 hour. It was then filtered and the filtrate was concentrated to give 2.07 g white solid (100%).

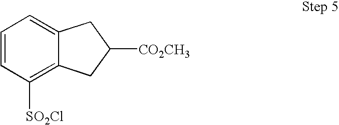

Methyl 4-chlorosulfonyl-indan-2-carboxylate: A mixture of methyl-4-aminoindane-2-carboxylate (2.5 g, 0.013 mol), 12.5 mL acetonitrile, and 12.5 mL H₂O was cooled to −5° C. in an ice-salt bath. To this was added 2.6 mL concentrated HCl (0.014 mol). To this was added dropwise over 20 minutes a solution of 1.0 g sodium nitrite (0.021 mol) in 5 mL water. After the addition was complete the solution was stirred for 20 minutes. It was then transferred to a jacketed addition funnel cooled with ice water. The solution was added dropwise to a solution stirred under nitrogen at 55° C. of 4.2 g potassium thioxanthate (0.026 mol) in 20 mL H₂O. As the addition took place, a dark layer rose to the top of the diazonium ion solution which was not added. After the addition was complete the mixture was stirred at 55° C. for 30 minutes, then was allowed to cool and was extracted with 40 mL ethyl acetate. The organic layer was dried (MgSO₄) and concentrated. The residue was loaded on 80 mL silica gel which was slurry-packed in hexanes. This was eluted with 100 mL hexanes, then 1%-50% CH₂Cl₂ in hexanes in 50 mL fractions to give 1.3 g amber oil (33%).

A mixture of 3.6 g of the above compound in 30 mL CCl₄ and 10 mL H₂O was vigorously stirred and cooled to 3 C. Chlorine gas was bubbled through at such a rate that the temperature stayed below 10° C. After conversion was complete, the phases were separated and the aqueous layer was extracted with CH₂Cl₂. The combined organic layers were dried (MgSO₄) and concentrated to give 4.0 g yellow oil (100%).

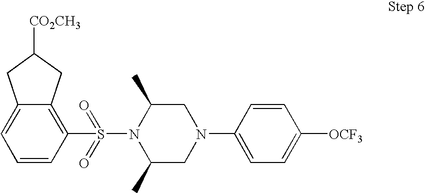

4-[cis-2,6-Dimethyl-4-(4-trifluoromethoxy-phenyl)-piperazine-1-sulfonyl]-indan-2-methyl ester: A mixture of methyl 4-chlorosulfonyl-indan-2-carboxylate (2.13 g, 0.0078 mol) obtained from Step 6, cis-3,5-dimethyl-1-(4-trifluoromethoxy-phenyl)-piperidine (3.0 g, 0.0109 mol) obtained from Example 51, 20 mL acetonitrile, and 3.0 g K₂CO₃ (0.0217 mol) was heated to 60° C. under nitrogen with stirring for 20 hours. It was then filtered and the filtrate was concentrated. The residue was purified by chromatography on silica (5%-50% EtOAc in hexanes) to give 2.64 g viscous yellow oil (66%).

### Step 7

4-[cis-2,6-Dimethyl-4-(4-trifluoromethoxy-phenyl)-piperazine-1-sulfonyl]-indan-2-carboxylic acid: To a solution of 4-[cis-2,6-dimethyl-4-(4-trifluoromethoxy-phenyl)-piperazine-1-sulfonyl]-indan-2-methyl ester (2.64 g, 0.0052 mol) in the minimum amount of THF (ca 15 mL) was added a solution of 0.14 g LiOH (0.0057 mol) in the minimum amount of water (ca 2.5 mL). This was capped and stirred at room temperature for 12 hours. Examination by HPLC showed the reaction was 85% complete so an additional 0.020 g LiOH (0.125 eq total) was added and stirring was continued for 3 hours. It was then concentrated to remove THF and partitioned between EtOAc and water. The aqueous layer was treated with 0.54 mL conc. HCl. It was then extracted with ethyl acetate. The organic layer was dried (MgSO₄) and concentrated to give 2.38 g yellow amorphous solid (93%).

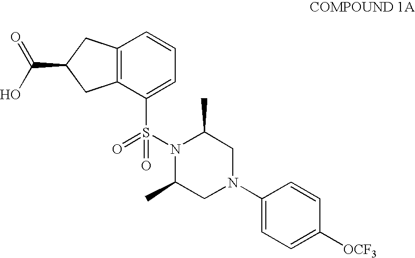

### (S)-4-[cis-2,6-Dimethyl-4-(4-trifluoromethoxy-phenyl)-piperazine-1-sulfonyl]-indan-2-carboxylic acid

A single enantiomer of Compound 1 was obtained by chiral HPLC (chiralpak ASH 0.46×15 cm Hex/IPA 94:6 (v/v) with 0.1% TFA, flow rate 1 ml/min) separation from the racemate. LCMS 497.1 (M−1)⁻¹.

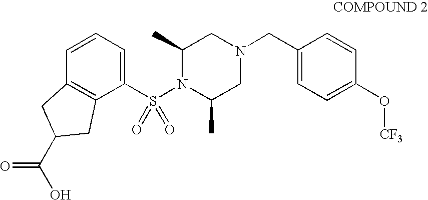

### 4-[cis-2,6-Dimethyl-4-(4-trifluoromethoxy-benzyl)-piperazine-1-sulfonyl]-indan-2-carboxylic acid

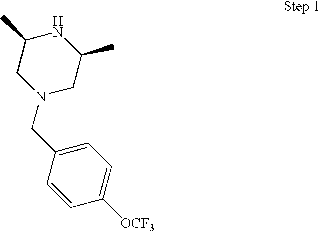

cis-3,5-Dimethyl-1-(4-trifluoromethoxy-benzyl)-piperazine: To a solution of 4-(trifluoromethoxy)-benzaldehyde (776 uL, 4.38 mmol) in methylene chloride (30 mL) was added cis-2,6-dimethyl piperazine (1.0 g, 8.77 mmol). After 1 hour sodium triacetoxy borohydride (2.45 g, 8.77 mmol) was added to the mixture. The solution was stirred at room temperature for an additional 4 hours. The reaction was concentrated in vacuo, diluted with ethyl acetate and extracted with 1N HCl (2×50 mL). The aqueous layer was then neutralized with NaOH and extracted with ethyl acetate (3×50 mL). The organic layer was dried (Na₂SO₄) and concentrated to provide cis-3,5-dimethyl-1-(4-trifluoromethoxy-benzyl)-piperazine (1.01 g, 80%). ¹H NMR (400 MHz, CD₃OD) δ 7.42 (d, 2H), 7.23 (d, 2H), 3.54 (s, 2H), 2.98-2.88 (m, 2H), 2.82-2.74 (m, 2H), 1.69 (t, 2H), 1.05 (d, 6H); LCMS 289.5 (M+1)⁺.

### Step 2

4-[cis-2,6-Dimethyl-4-(4-trifluoromethoxy-benzyl)-piperazine-1-sulfonyl]-indan-2-carboxylic acid: The compound 4-[cis-2,6-dimethyl-4-(4-trifluoromethoxyl-benzyl)-piperazine-1-sulfonyl]-indan-2-carboxylic acid was synthesized according to the procedure for Compound 1 using cis-3,5-dimethyl-1-(4-trifluoromethoxy-benzyl)-piperazine. ¹H NMR (400 MHz, CD₃OD) δ 7.74-7.64 (m, 4H), 7.47 (d, 1H), 7.39-7.28 (m, 2H), 4.42 (s, 2H), 4.21-2.18 (m, 2H), 3.50-3.34 (m, 5H), 3.33-3.19 (m, 4H), 1.56 (d, 6H); LCMS 497.5 (M+1)⁺.

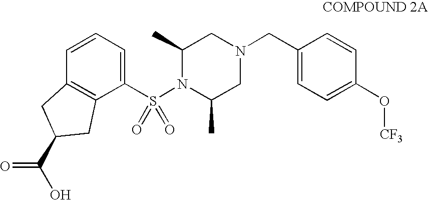

### (S)-4-[cis-2,6-Dimethyl-4-(4-trifluoromethoxy-benzyl)-piperazine-1-sulfonyl]-indan-2-carboxylic acid

The product from Compound 2 Step 1 and the product from Compound 1 Step 5 were reacted using the conditions outlined in Compound 1 Step 6 to yield the racemic methyl ester. Chiral separation using OJ-H, 25% methanol in CO₂ (100 bar), 5 mL/min followed by hydrolysis using conditions outlined in Compound 1 Step 7 provided a single enantiomer of 4-(cis-2,6-dimethyl-4-(3-trifluoromethoxy)benzyl)piperazin-1-ylsulfonyl)-2,3-dihydro-1H-indene-2-carboxylic acid. ¹H NMR (400 MHz, CD₃OD) δ 7.66 (d, 1H), 7.46 (d, 1H), 7.41 (d, 2H), 7.36-7.30 (m, 1H), 7.19 (d, 2H), 4.08-3.99 (m, 1H), 3.94-3.8 (m, 1H), 3.56-3.49 (m, 2H), 3.43 (s, 2H), 3.40-3.22 (m, 3H), 2.57 (t, 2H), 2.09-1.92 (m, 2H), 1.56 (d, 6H); LCMS 513.5 (M+1)⁺.

### Preparation of Tosylate Salt of Compound 1A

### Step 1

32% HCl is added to a solution of sodium nitrite in water and acetonitrile at 0° C. The solution is cooled to −5° C. and a solution of (R,S)-4-amino-indan-2-carboxylic acid methyl ester hydrochloride in water, acetonitrile, and 32% HCl is added, keeping the temperature between −7 and −10° C. The resulting cold diazonium solution is added to a solution of potassium ethylxanthogenate, in water and acetonitrile, at 60° C. After heating at 60° C., the mixture is cooled to room temperature and extracted from dichloromethane. The organic solution is charged into the reactor and concentrated under reduced pressure. Dichloromethane and water are added, the mixture cooled to 5° C., and chlorine gas passed through the mixture. The organic solution is separated and the aqueous solution is extracted from dichloromethane. The combined organic solution is dried over magnesium sulfate and concentrated under reduced pressure to afford (R,S)-4-chlorosulfonyl-indan-2-carboxylic acid. HPLC may be used to monitor the reaction.

### Step 2:

Potassium carbonate is added to a mixture of cis-3,5-dimethyl-1-(4-trifluoromethoxy-phenyl)-piperazine hydrochloride in dichloromethane and water. After stirring at room temperature, the organic phase is collected and the aqueous layer extracted from dichloromethane. The combined organic solution is charged into the reactor and concentrated under reduced pressure, followed by the addition of acetonitrile and potassium carbonate. A solution of (R,S)-4-chlorosulfonyl-indan-2-carboxylic acid, in acetonitrile, is added to the reaction mixture. After heating at 50° C., the reaction mixture is cooled to 20° C. The mixture is transferred into a 200 L movable agitation feed tank, which is charged with Celite, and the suspension is stirred. The suspension is filtered, filter cake washed with acetonitrile, and the filtrate is concentrated under reduced pressure, cooled to 0-5° C., and 32% HCl added. Following further concentration and filtration, the filtrate is concentrated to give an oil which is purified by silica gel chromatography and recrystallized from isopropanol to give the product (R,S)-4-[cis-2,6-dimethyl-4-(4-trifluoromethoxy-phenyl)-piperazine-1-sulfonyl]-indan-2-carboxylic acid methyl ester (>95% by HPLC).

### Step 3:

Simulated moving bed (SMB) chromatography was used to separate the S- and R-enantiomers of (R,S)-4-[cis-2,6-dimethyl-4-(4-trifluoromethoxy-phenyl)-piperazine-1-sulfonyl]-indan-2-carboxylic acid methyl ester. The SMB method uses a Chiralpak AS column and nheptane/isopropanol (1:1 v/v) to yield the S-enantiomer, (S)-4-[cis-2,6-dimethyl-4-(4-trifluoromethoxy-phenyl)-piperazine-1-sulfonyl]-indan-2-carboxylic acid methyl ester (>99.0% by chiral HPLC).

### Step 4:

To a solution of (S)-4-[cis-2,6-dimethyl-4-(4-trifluoromethoxy-phenyl)-piperazine-1-sulfonyl]-indan-2-carboxylic acid methyl ester, in THF, is added a solution of lithium hydroxide in water, which is stirred at 20° C. and concentrated under reduced pressure. The reaction mixture is cooled to 9° C., neutralized with 32% HCl, and extracted from toluene. Water is removed from the organic solution by azeotropic distillation. Following distillation, the organic solution is cooled to ambient temperature and transferred to a feeding vessel. The reactor is charged with p-toluenesulfonic acid in toluene and water is removed by azeotropic distillation. The solution is cooled to 60° C., followed by the addition of the organic solution from the feeding vessel. The mixture is stirred at 83° C., then cooled to 10° C. to induce crystallization. The product suspension is filtered, the filter cake rinsed with heptane, and dried on a rotovap, at 40° C., to afford (S)-4-[cis-2,6-dimethyl-4-(4-trifluoromethoxy-phenyl)-piperazine-1-sulfonyl]-indan-2-carboxylic acid tosylate. ¹HNMR δ 1.60 (d), 1.62 (d), 2.33 (s), 3.23 (m), 3.49 (m), 3.39 (m), 4.05 (m), 4.49 (m), 3.40 (dd), 3.23 (dd), 7.14 (d), 7.14 (d), 7.09 (d), 7.09 (d), 7.59 (d), 7.59 (d), 7.71 (d), 7.26, dd, 7.57 (d), 7.57 (d), 7.40 (d).

### Several Exemplary Oral Formulations of Compound 1A

Total fill weight of each capsule is 300 mg, not including capsule weight. Target compound dosage as free base is 1, 5, or 20 mg per capsule, thereby allowing for a range of dosages to be conveniently administered to a patient. Several exemplary dosages are set forth below in Table 1.

TABLE 1
Exemplary Formulations

| | Capsule Target Dosage, | | |
|---|---|---|---|
| | Compound 1A | | |
| | 1 mg | 5 mg | 20 mg |
| Capsule Color | white | green | orange |
| Compound 1A tosylate, mg | 1.35 | 6.73 | 26.91 |
| Lactose monohydrate, mg | 277.65 | 272.27 | 252.09 |
| Silicon dioxide, mg | 3 | 3 | 3 |
| Crospovidone, mg | 15 | 15 | 15 |
| Magnesium stearate, mg | 3 | 3 | 3 |
| (vegetable grade) | | | |
| Total Fill Weight, mg | 300 | 300 | 300 |

The capsules above may be made by the following methods.

### Blending and Capsule Fill for Compound 1A Capsule 20 mg

Compound 1A is passed between two sieve screens to produce a consistent particle size in the range of 53 to 250 μm. Particle size and XRPD in-process control (IPC) testing are performed, for information only, to profile the particle size distribution and evaluate the solid-state characteristics of Compound 1A following the sieve step. An appropriate amount of Compound 1A is weighed for each batch. The excipients are passed through a 250 μm sieve, collected, and weighed. Approximately half of the excipients are added to the container for blending. Compound 1A is added to the container, followed by the remaining half of the excipients, such that Compound 1A is sandwiched between the excipients. The blend is then mixed until uniform. Blend uniformity IPC (in-process control) testing is performed after blending by sampling 3 points within the container (top, middle, and bottom) and testing each sample for potency. A test result of 95-105% of target, with an RSD of 5%, must be achieved before the process can continue. Additional blend time is allowed to achieve a uniform blend if the IPC test results are not within the specified range. Upon acceptable blend uniformity results, a measured aliquot of the Compound 1A stock formulation is separated to manufacture the lower strengths. This aliquot is removed from the manufacturing area. Magnesium stearate is passed through a 75 μm sieve, collected, weighed, added to the blender as a lubricant, and mixed until dispersed. The final blend is weighed and reconciled. The Coni-Snap capsules (Swedish orange) are opened and the body of the capsule is placed on a Profill tray (holding 100 units) and the cap is placed on the corresponding tray. Blended materials are then flood fed into the body of the capsules using a spatula. The trays may be tamped to settle the blend in each capsule to assure uniform target fill weight. The capsules are then sealed by combining the filled bodies with the caps. Fill weight uniformity IPC testing is performed following encapsulation. Thirteen capsules are removed from a tray of 100 and weighed. This test result must be within the target fill weight (376±15 mg, including capsule weight) to pass. If the target fill weight specification is not met, the entire tray of 100 is weight checked and capsules not meeting weight specification are rejected. Following successful fill weight check, capsules are inspected, de-dusted, reconciled, and placed into a suitable in-process storage container.

### Blending and Capsule Fill for Compound 1A, Capsule 5 mg and Capsule 1 mg

Compound 1A stock formulation aliquot can be used to manufacture the lower strengths using serial dilutions. The excipients are passed through a 250 μm sieve, collected, and weighed. Approximately half of the excipients are added to the container for blending. Compound 1A stock formulation aliquot is added to the container, followed by the remaining half of the excipients, such that Compound 1A stock formulation aliquot is sandwiched between the excipients. The blend is then mixed until uniform. Blend uniformity IPC testing is performed after blending by sampling 3 points within the container (top, middle, and bottom) and testing each sample for potency. A test result of 95-105% of target, with an RSD of 5%, must be achieved before the process can continue. Additional blend time is allowed to achieve a uniform blend if the IPC test results are not within the specified range. Upon acceptable blend uniformity results, an aliquot of Compound 1A stock formulation can be removed to manufacture the 1 mg strength. This aliquot is removed from the manufacturing area.

Magnesium stearate is passed through a 75 μm sieve, collected, weighed, and added to the blender as a lubricant and mixed until dispersed. The final blend is weighed and reconciled. The Coni-Snap capsules (5 mg, dark green or 1 mg, white) are opened and the body of the capsule is placed on a Profill tray (holding 100 units) and the cap is placed on the corresponding tray. Blended materials are then flood fed into the body of the capsules using a spatula. The trays may be tamped to settle the blend in each capsule to assure uniform target fill weight. The capsules are then sealed by combining the filled bodies with the caps. Fill weight uniformity IPC testing is performed following encapsulation. Thirteen capsules are removed from a tray of 100 and weighed. This test result must be within the target fill weight (376±15 mg, including capsule weight) to pass. If the target fill weight specification is not met, the entire tray of 100 is weight checked and capsules not meeting weight specification are rejected. Following successful fill weight check, capsules are inspected, de-dusted, reconciled, and placed into a suitable in-process storage container.

### Biological Activity

All compounds listed as Examples above are known PPARδ modulators. The activity of these and other compounds as PPARδ modulators is demonstrated in PCT/US2004/010737, filed on Apr. 7, 2004; PCT/US2004/010889, filed on Apr. 7, 2004; PCT/US2004/010970, filed on Apr. 7, 2004; U.S. application Ser. No. 10/820,647, filed Apr. 7, 2004; PCT/US2004/043031, filed Dec. 20, 2004; PCT/US2005/011751 filed Apr. 7, 2005; U.S. application Ser. No. 11/102,356 filed Apr. 7, 2005; and U.S. application Ser. No. 11/258,463, filed Oct. 25, 2005; the contents of all of which are hereby incorporated by reference as if written herein in their entirities.

### In Vivo Assay

### Evaluation of Pharmacological Efficacy of Compound 1 of the Present Invention in Hyperlipidemic Rhesus Macaques

Rhesus macaques share great genetic similarity with humans, and can naturally develop the same metabolic disorders having equivalent phenotypic hallmarks as those found in humans. (Hansen, B C: Presentation at the IBC Second International Conference, 5-4-2004). For example, both humans and nonhuman primates can develop metabolic syndrome, a suspected prodrome to diabetes mellitus, cardiovascular disease and other life-threatening conditions.

Metabolic syndrome (alternately known by metabolic syndrome X, multiple risk factor syndrome, insulin resistance syndrome, plurimetabolic syndrome, diabesity, etc.) can be defined in both humans and non-human primates by measured increases in plasma LDL, triglycerides (TG), and plasma glucose, and a decrease in plasma HDL. In humans, metabolic syndrome is defined by the World Health Organization (WHO) as insulin resistance plus 2 of the following criteria: plasma TG ≧150 mg/dL, HDL <35 mg/dL (M); <40 mg/dL (W), body mass index (BMI) >30, elevated blood pressure (BP) ≧140/90 or drug R^{x}, and urinary albumin >20 mg/min. Similar, alternative measures are defined by the National Cholesterol Education Program (NCEP). Comparable indices exist for the diagnosis of metabolic syndrome, and indeed, for may other disorders, in nonhuman primates (Hansen et al.). The similarities between humans and non-human primates such as rhesus macaques make the macaques ideal models for studying the biological mechanisms of metabolic and cardiovascular diseases and for identifying in preclinical trials candidates for pharmaceutical treatments for these disorders.

In the present study, n=6 male obese rhesus macaques were selected for the study and prepared with 4 weeks of diet run-in (week −4 to week 0). At week 0, blood samples were taken in order to determine baseline plasma levels of CRP were measured prior to beginning dosage with Compound 1 as tosylate salt. Dosage began on the same day at 6 mg/kg/day in divided doses, given with food, and continued for four weeks. Dose was subsequently escalated at 4-week intervals to 12 mg/kg/d, 24 mg/kg/d, and finally 48 mg/kg/day. Plasma samples were taken at the close of each 4-week regimen, and a final sample was taken after 4 weeks of washout following discontinuation of dosing.

Quantitative measurements of plasma CRP in each sample taken were made by LabCorp, Inc. (Laboratory Corporation of America) by a multi-calibrator based high sensitivity wide ranged method using a immunoturbidometric assay. The limit of quantitation for the assay was 0.05 mg/dL and the highest point on the calibrator was 32 mg/dL. Mean values as percent change from baseline, and standard deviation as percent change from baseline, were calculated across the group and are shown in Table 2. Mean values for weeks 8-16 fell below the limit of quantiatation, and no data could therefore be reported for the standard deviation.

TABLE 2

| | | | Plasma CRP |
|---|---|---|---|
| | Dose, mg/kg | Mean Plasma | Standard Deviation, |
| Week | per day | CRP, ng/mL | +/− in ng/mL |
| 0 (diet run-in) | 0 | 0.233 | 0.103 |
| 4 | 6 | 0.166 | 0.115 |
| 8 | 12 | <0.05 | n/a |
| 12 | 24 | <0.05 | n/a |
| 16 | 48 | <0.05 | n/a |
| 20 (washout) | 0 | 0.183 | 0.116 |

Table 3 shows that plasma CRP was significantly reduced in hyperlipidemic rhesus macaques over 16 weeks of dose-escalating treatment with Compound 1. Most interestingly, by week 8, CRP levels were below detectable levels (LOQ=0.05 ng/mL) and remained undetectable throughout the rest of the dosing regimen. During the washout period, the CRP levels reverted back to the pre-treatment levels implying that these changes are treatment-related.

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A method of reducing CRP comprising the selective modulation of PPARδ.

2. The method as recited in claim 1 wherein said selective modulation is 100-fold or greater for PPARδ over PPARα sand PPARγ.

3. The method as recited in claim 1 wherein said modulation comprises contacting said PPARδ with a compound of structural Formula I: 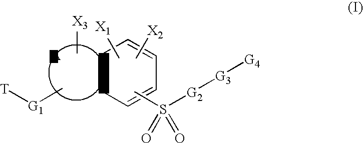
or a salt, ester, or prodrug thereof, wherein:
A is a saturated or unsaturated hydrocarbon chain or a heteroatom-comprising hydrocarbon chain having from 3 to 5 atoms, forming a five- to seven-membered ring;
T is selected from the group consisting of —C(O)OH, —C(O)NH₂, and tetrazole;
G₁ is selected from the group consisting of —(CR¹R²)ₙ—, -Z(CR¹R²)ₙ—, —(CR¹R²)ₙZ-, —(CR¹R²)ᵣZ(CR¹R²)ₙ—,
Z is O, S or NR;
n is 0, 1, or 2;
r and s are independently 0 or 1;
R¹ and R² are independently selected from the group consisting of hydrogen, halo, optionally substituted lower alkyl, optionally substituted lower heteroalkyl, optionally substituted lower alkoxy, and lower perhaloalkyl or together may form an optionally substituted cycloalkyl;
X₁, X₂, and X₃ are independently selected from the group consisting of hydrogen, optionally substituted lower alkyl, optionally substituted cycloalkyl, halogen, perhaloalkyl, hydroxy, optionally substituted lower alkoxy, nitro, cyano, and NH₂;
G₂ is selected from the group consisting of a saturated or unsaturated cycloalkyl or heterocycloalkyl linker, optionally substituted with X₄ and X₅;
X₄ and X₅ are independently selected from the group consisting of hydrogen, optionally substituted lower alkyl, halogen, lower perhaloalkyl, hydroxy, optionally substituted lower alkoxy, nitro, cyano, NH₂, and CO₂R, or X₄ and X₅ together may form a carbocycle;
R is selected from the group consisting of optionally substituted lower alkyl and hydrogen;
G₃ is selected from the group consisting of a bond, a double bond, —(CR³R⁴)ₘ—, carbonyl, and —(CR³R⁴)ₘCR³═CR⁴—;
m is 0, 1, or 2;
R³ and R⁴ are independently selected from the group consisting of hydrogen, optionally substituted lower alkyl, optionally substituted lower alkoxy, optionally substituted aryl, lower perhaloalkyl, cyano, and nitro;
G₄ is selected from the group consisting of hydrogen, optionally substituted aryl, optionally substituted heteroaryl, optionally substituted cycloalkyl, optionally substituted cycloheteroalkyl, optionally substituted cycloheteroaryl, optionally substituted cycloalkenyl, and —N═(CR⁵R⁶); and
R⁵ and R⁶ are independently selected from the group consisting of hydrogen, optionally substituted alkyl, optionally substituted aryl, optionally substituted heteroaryl, optionally substituted cycloalkyl, optionally substituted cycloalkenyl, and optionally substituted cycloheteroalkyl.

4. The method as recited in claim 3 wherein the compound has the structural formula 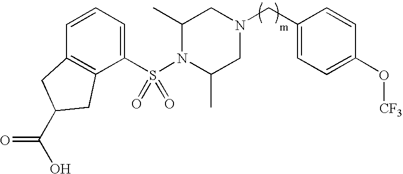
and m is 0 or 1.

5. The method as recited in claim 4 wherein said compound is selected from the group consisting of Compound 1, Compound 1A, Compound 3, and Compound 3A.

6. The method as recited in claim 3 wherein said compound is Compound 1A.

7. A method of reducing CRP comprising the administration of a therapeutically effective amount of (S)-4-[cis-2,6-dimethyl-4-(4-trifluoromethoxy-phenyl)-piperazine-1-sulfonyl]-indan-2-carboxylic acid tosylate.

8. The method as recited in claim 7 wherein said effective amount is greater than 20 mg per day when dosed orally.

9. The method as recited in claim 8 wherein said amount is between about 20 and about 140 mg per day when dosed orally.

10. The method as recited in claim 9 wherein said effective amount, when dosed orally, is selected from the group consisting of: about 20 mg per day, about 40 mg per day, about 60 mg per day, about 80 mg per day, and about 100 mg per day.

11. A pharmaceutical composition comprising:
a) a selective PPARδ modulator;
b) an amount of a folate effective to reduce CRP; and
c) together with one or more pharmaceutically acceptable active ingredients or adjuvants.

12. The pharmaceutical composition of claim 11 wherein said folate is folic acid.

13. A method of treating a patient comprising:
a. determining CRP levels of said patient;
b. determining if said CRP levels are too high for said patient; and
c. if said CRP levels are too high, having said patient take a therapeutically effective amount of a selective PPARδ modulator.
